(19) United States
(12) Patent Application Publication
(10) Pub. No.: US 2019/0000529 A1
(43) Pub. Date: Jan. 3, 2019

(54) SYSTEM FOR CONTROLLING ACTIVATION OF MULTIPLE APPLICATORS FOR TISSUE TREATMENT

(71) Applicant: Cutera, Inc., Brisbane, CA (US)

(72) Inventors: Amogh KOTHARE, Fremont, CA (US); Lukas HUNZIKER, San Jose, CA (US); Cindy HSIEH, Los Altos, CA (US); Brian ATHOS, Brisbane, CA (US)

(73) Assignee: Cutera, Inc., Brisbane, CA (US)

(21) Appl. No.: 16/022,396

(22) Filed: Jun. 28, 2018

Related U.S. Application Data
(60) Provisional application No. 62/526,214, filed on Jun. 28, 2017.

(51) Int. Cl. A61B 18/14

(52) U.S. Cl. CPC A61B 18/1402; A61B 2018/00714

(57) ABSTRACT

Systems and methods for applying energy to treat body areas having fat deposits, cellulite, or loose skin are disclosed. The treatment energy is applied to a patient with multiple applicators in contact with the patient's skin, which heats the skin and underlying tissue, such as fat. As the temperature of the fat is raised and maintained for a period of time, the heat damages the fat cells. When multiple applicators apply energy to multiple treatment subareas within a general area of a patient's body at interleaving intervals, treatment efficiency is improved. In particular, compared to applying energy continuously to treat each subarea one at a time, applying energy in interleaving intervals sequentially to the various subareas reduces the total treatment time by having multiple subareas treated simultaneously while maintaining the temperature of the target tissue (e.g. fat) within the therapeutic temperature range.

### CROSS REFERENCE TO RELATED APPLICATIONS

This application claims the priority benefit of U.S. Provisional Application Ser. No. 62/526,214 filed Jun. 28, 2017, which is incorporated herein by reference in its entirety.

### BACKGROUND

The present disclosure relates to systems and methods for applying energy (e.g., electromagnetic radiation including visible light, infrared light (such as heat energy), radio waves, and/or microwaves, as well as electricity and/or ultrasound) to treat, for example, body areas having fat deposits, cellulite, or loose skin. The treatment energy is applied to the patient with an applicator that contacts the patient's skin, which heats the skin and underlying tissue, such as fat. As the temperature of the fat is raised and maintained for a period of time, the heat damages the fat cells. By applying energy in accordance with a manner designed to raise and maintain the temperature of the fat tissue, a clinician is able to selectively target areas of a patient's body, resulting in reducing fat tissue in those areas.

In some cases, it may be desirable to have multiple applicators applying energy to multiple treatment subareas within a general area of a patient's body at different, interleaving intervals in order to improve the treatment efficiency. In particular, compared to applying energy continuously to treat each subarea one at a time, applying energy in interleaving intervals sequentially to the various subareas reduces the total treatment time by having multiple subareas treated simultaneously while maintaining the temperature of the target tissue (e.g. fat) within the therapeutic temperature range. Furthermore, compared to applying energy continuously to all subareas, applying energy in interleaving intervals sequentially to the various subareas generates minimal discomfort to the patient. Thus, interleaving and multiplexing the application of energy to multiple subareas is a technique designed to energize more than a single applicator without sacrificing treatment time, efficacy, or patient comfort.

### BRIEF SUMMARY

The following presents a simplified summary of one or more examples in order to provide a basic understanding of such examples. This summary is not an extensive overview of all contemplated examples, and is intended to neither identify key or critical elements of all examples nor delineate the scope of any or all examples. Its purpose is to present some concepts of one or more examples in a simplified form as a prelude to the more detailed description that is presented below.

Systems and methods for treating an area of a patient comprising a plurality of subareas with energy are disclosed. The treatment system comprises one or more energy sources, wherein each energy source is configured to independently provide radiofrequency energy; a plurality of energy applicators, numbering more than the number of energy sources, wherein each energy applicator is aligned with a different subarea and is configured to apply energy to the subarea when provided with energy from the one or more energy sources; and a switching circuit configured to energize each energy applicator in the plurality of energy applicators with energy provided from the one or more energy sources using a predetermined pattern of energization. The predetermined pattern of energization comprises: a first phase lasting a first time period, wherein the energy sources sequentially provide energy to multiple applicators one or more times at a frequency and a first range of power levels to elevate temperatures of fat tissue in each subarea to a fat treatment temperature, wherein the temperature of fat tissue in a subarea does not fall more than 2 degrees Celsius during any time in the first time period when energy is not being applied to the subarea; and a second phase lasting a second time period, wherein the energy sources sequentially and repeatedly provide energy to multiple applicators at a frequency and at a second range of power levels to maintain temperatures of fat tissue in each subarea at or above the fat treatment temperature, wherein the temperature of fat tissue in a subarea does not fall more than 2 degrees Celsius during any time in the second time period when energy is not being applied to the subarea.

In some embodiments, the temperature of fat tissue in a subarea does not fall more than a threshold temperature drop, such as 1 degree Celsius or 0.5 degree Celsius, during any time in the first time period when energy is not being applied to the subarea. In some embodiments, during the first time period, the time between consecutive applications of energy to each energy applicator is less than a certain time threshold, such as 180 seconds, 120 seconds, or 60 seconds. In some embodiments, during the second time period, the time between consecutive applications of energy to each energy applicator is less than another certain time threshold, such 60 seconds, 45 seconds, or 30 seconds.

In some embodiments, the plurality of applicators are grouped into 3 pairs of applicators, the treatment area of the patient comprises 6 subareas, each of 6 energy applicators is applied to each of the 6 subareas, the first phase comprises repeatedly and sequentially applying energy to each pair of applicators, and the second phase comprises repeatedly and sequentially applying energy to each pair of applicators. In some embodiments, a first energy source is applied to the first of each pair of applicators; a second energy source is applied to the second of each pair of applicators; and the first energy source is between 170 degrees and 190 degrees out of phase with the second energy source. In some embodiments, the first energy source is 180 degrees out of phase with the second energy source.

In some embodiments, one energy applicator of the pair of energy applicators is electrically connected as the current return path of the other energy applicator of the pair of energy applicators. In some embodiments, the energy applicators in each pair of energy applicators are not adjacent to each other. In some embodiments, the first time period is between 20 and 225 seconds. In some embodiments, the second time period is between 9 minutes and 15 minutes.

In some embodiments, the frequency of the energy sources is within a range such as between 200 kHz and 10 MHz, between 1 MHz and 6.5 MHz, or between 1 MHz and 3 MHz, or is about 2 MHz. In some embodiments, the fat treatment temperature is between 43 degrees Celsius and 47 degrees Celsius. In some embodiments, each subarea has a surface area between 20 square cm and 80 square cm. In some embodiments, the second time period is within a range such as between 6 minutes and 25 minutes or between 8 minutes and 20 minutes.

### BRIEF DESCRIPTION OF THE FIGURES

The patent or application file contains at least one drawing executed in color. Copies of this patent or patent application publication with color drawing(s) will be provided by the Office upon request and payment of the necessary fee.

For a better understanding of the various described examples, reference should be made to the description below, in conjunction with the following figures in which like reference numerals refer to corresponding parts throughout the figures.

FIG. 1 illustrates an exemplary treatment device.

FIG. 2 illustrates an example switching diagram for a treatment system that applies energy to a patient with multiple applicators.

FIG. 3 illustrates an example circuit diagram for use with an applicator.

FIG. 4 illustrates an example switching diagram for the treatment system when energy is flowing through the system.

FIG. 5A illustrates an example energization pattern of how energy may be provided to three different applicators.

FIG. 5B illustrates an example energization pattern of how energy may be alternately provided to six different applicators.

FIG. 6 illustrates another example switching diagram for the treatment system when energy is flowing through the system.

FIGS. 7A and 7B illustrate different arrangements of applicators that can be used when applying energy to treatment areas.

FIG. 8 illustrates an example energization pattern of how energy may be provided to three pairs of applicators.

FIG. 9 illustrates another example switching diagram for the treatment system when energy is flowing through the system.

FIG. 10 illustrates an example energization pattern of how energy may be provided simultaneously to different pairs of applicators.

FIG. 11 illustrates an exemplary interleaving energization pattern with a constant duty cycle and variable individual application time.

FIG. 12 illustrates the fat temperature progression during treatment using the interleaving energization pattern with a constant duty cycle and variable individual application time when performed in an in vivo experiment.

FIG. 13 illustrates an exemplary interleaving energization pattern with a variable duty cycle and variable individual application time.

FIG. 14 illustrates an exemplary interleaving energization pattern with a constant duty cycle and constant individual application time.

FIG. 15A illustrates the reduction in the fat layer thickness for various therapeutic exposure times as measured in a clinical study.

FIG. 15B illustrates the occurrence rate and duration of nodule formation in the fat layer for various therapeutic exposure times as measured in a clinical study.

### DETAILED DESCRIPTION

The detailed description set forth below in connection with the appended drawings is intended as a description of various configurations and is not intended to represent the only configurations in which the concepts described herein can be practiced. The detailed description includes specific details for the purpose of providing a thorough understanding of various concepts. However, it will be apparent to those skilled in the art that these concepts can be practiced without these specific details. In some instances, well-known structures and components are shown in block diagram form in order to avoid obscuring such concepts.

Examples of systems and methods for controlling activation of multiple applicators for tissue treatment will now be presented with reference to various electronic devices and methods. These electronic devices and methods will be described in the following detailed description and illustrated in the accompanying drawing by various blocks, components, circuits, steps, processes, algorithms, etc. (collectively referred to as “elements”). These elements can be implemented using electronic hardware, computer software, or any combination thereof. Whether such elements are implemented as hardware or software depends upon the particular application and design constraints imposed on the overall system.

By way of example, an element, or any portion of an element, or any combination of elements of the various electronic systems can be implemented using one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionalities described throughout this disclosure. One or more processors in the processing system can execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more examples, the functions described for the system for controlling activation can be implemented in hardware, software, or any combination thereof. If implemented in software, the functions can be stored on or encoded as one or more instructions or code on a computer-readable medium. Computer-readable media can include transitory or non-transitory computer storage media for carrying or having computer-executable instructions or data structures stored thereon. Both transitory and non-transitory storage media can be any available media that can be accessed by a computer as part of the processing system. By way of example, and not limitation, such computer-readable media can include a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer-executable code in the form of instructions or data structures accessible by a computer. Further, when information is transferred or provided over a network or another communications connection (either hardwired, wireless, or combination thereof) to a computer, the computer or processing system properly determines the connection as a transitory or non-transitory computer-readable medium, depending on the particular medium. Thus, any such connection is properly termed a computer-readable medium. Combinations of the above should also be included within the scope of the computer-readable media. Non-transitory computer-readable media exclude signals per se and the air interface.

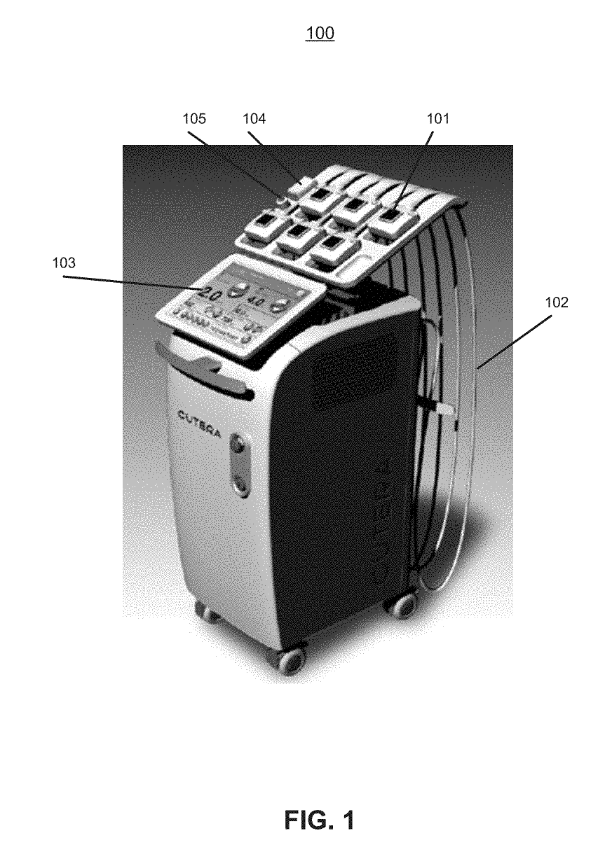

FIG. 1 illustrates a tissue treatment device 100. The tissue treatment device 100 includes a console that is configured to carry one or more energy sources. One or more applicators are connected to the console by one or more cords 102 that are configured to carry energy and/or communication signals to the applicators. The applicators apply energy to the patient's tissue. Some applicators, such as 101, may be designed to support use in a “hands-free” mode. In this mode, the applicators are held in place against the skin surface through a suitable means during the duration of the treatment. The handpieces may also be designed to support use in a “hand held” mode. In this mode, the operator holds the handpiece against the skin surface. In some embodiments, other applicators, such as 104, may be designed to be used only in the hand held mode. A touch screen interface, such as 103, is configured to allow a user to select which handpieces are used for a given treatment. For hands-free mode operation, a patient comfort switch 105 may be used to allow the patient to terminate treatment if it exceeds his or her comfort level.

In some embodiments, each applicator contains a temperature sensor that senses the skin temperature. In such cases, a control algorithm controls the energy delivery for each applicator to ramp the skin to a target temperature and then maintains the temperature in steady state, which is followed by a therapeutic (“therapy”) period during which the target tissue is selectively damaged by exceeding the threshold temperature during apoptosis or other mechanisms, such as hyperthermia. When targeting subcutaneous tissues such as fat, the known correlation between skin and fat temperatures is used to control the energy delivery such that the threshold temperature for fat is exceeded. In some embodiments, the tissue treatment device 100 is configured to allow the user to set target temperatures for each applicator independently. This feature is useful when the various applicators are applied to treatment subareas that have different thicknesses of fat or require different levels of treatment. In some embodiments, the user may view and/or change one or more of the target temperatures before and/or during treatment.

Embodiments of the present application provide a mechanism for controlling the activation of multiple applicators. The simplest approach would be to activate each applicator sequentially. In this case, energy is applied continuously for a single, fixed period to each applicator for the time needed to complete the treatment. Scaling a medical treatment to multiple applicators using this approach can be straightforward since the temperature response of tissues to continuous exposure is typically well understood and not difficult to control. However, sequential activation causes the total treatment time to scale with the number of applicators. For applications where large surface areas are treated (e.g. non-invasive body sculpting), many applicators may be needed to cover the entire treatment area. Therefore, continuous mode, sequential activation may significantly extend the total treatment time, which may be undesirable for the patient and the physician. Alternatively, the applicator size may be increased to cover the same area with fewer applicators. However, a large applicator size typically reduces its versatility in terms of localizing treatment to target areas as well as its ability to accommodate a wide range of body types and locations. A need exists, therefore, for a device that is configured to target a large area with multiple applicators employing a method where the treatment time is independent of the number of applicators and yet achieves the same degree of selective tissue damage and efficacy provided by continuous mode energy delivery.

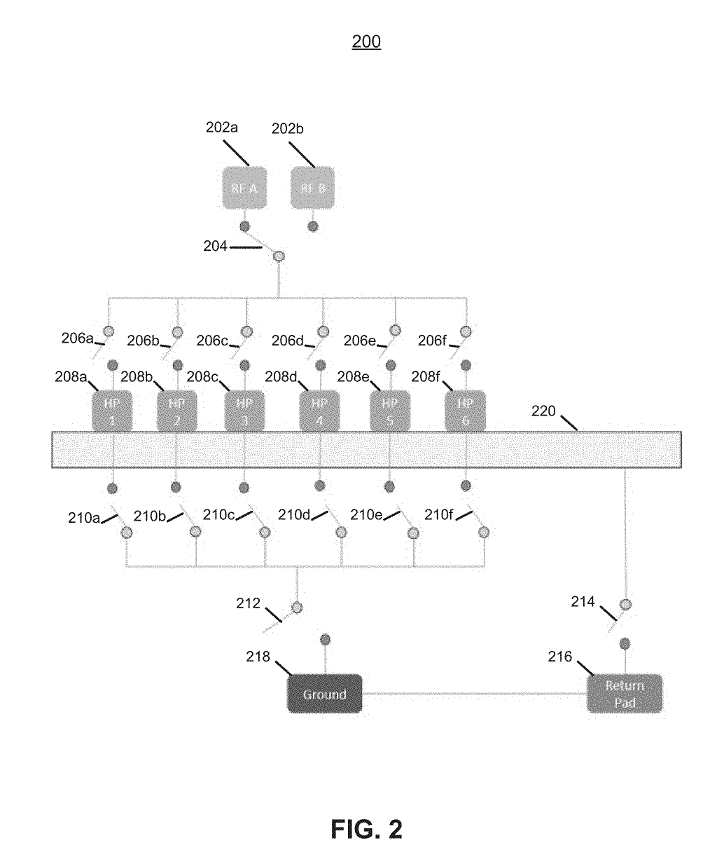

FIG. 2 illustrates an example switching diagram for a treatment system 200 that applies energy to a patient with multiple applicators 208a-208f (also referred to herein as “handpieces” or “HP”). The applicators 208a-208f (“HP 1” through “HP 6”) are attached to different treatment areas of the patient's body 220 and are configured to apply energy through the patient's skin to subcutaneous fat tissue. In some embodiments, the shape of the contact surface of an applicator is approximately a square. In some embodiments, the area of the contact surface of an applicator is between 20 cm² and 80 cm², with an area of approximately 40 cm² being preferred. As shown in FIG. 2, the system includes two energy sources 202a and 202b (“RF A” and “RF B”). The energy sources 202a and 202b may generate energy in the radio frequency (RF) spectrum. In some embodiments, each energy source 202a and 202b generates energy with a different frequency. The frequency affects the heating rate when the energy is applied to various tissues as well as the temperature differential between different tissues, such as skin and fat. For example, at least in the frequency range 200 kHz to 10 MHz, fat may reach a higher temperature than skin when experiencing the same applied energy source due to differences in RF attenuation coefficients and thermal properties of fat and skin. While operation within this range provides favorable treatment conditions more favorable treatment outcomes may result by further limiting the frequency range, depending on goal of the treatment. For example, increasing the frequency from 1 MHz to 2 MHz increases the temperature differential between fat and skin by 3° C. for typical treatment conditions. In some embodiments, the frequency is between 200 kHz and 10 MHz. In some embodiments, a frequency between 1 MHz and 6.5 MHz is more preferred. In some embodiments, the frequency is between 1 MHz and 3 MHz. In preferred embodiments, the frequency is approximately 2 MHz.

Each applicator 208a-208f can be electrically connected to either energy source 202a or 202b by selecting an energy source 202a or 202b with a source switch 204 and closing a corresponding applicator switch 206a-206f. In this way, each applicator 208a-208f is individually connectable to either energy source 202a or 202b. When one or more of the applicators 208a-208f are electrically connected to one of the energy sources 202a-202b, the energy then flows from the connected applicators 208a-208f, through the patient's body 220, to ground 218. The patient's body may be electrically connected to ground through one or more of the applicators 208a-208f that are not electrically connected to an energy source 202a-202b, or through a separate return pad 216 attached to the patient's body 220. Each of the applicators 208a-208f can be electrically connected to ground 218 by closing a corresponding return switch 210a-210f and ground switch 212. Alternatively, the patient's body 220 can be electrically connected to ground 218 by closing a return pad switch 214.

In some embodiments, each applicator switch 206a-206f is implemented with its corresponding return switch 210a-210f as a single switch (e.g. SP4T) so that no handpiece can be attached to both an RF source as well as ground 218 simultaneously. This safety feature prevents the RF sources from shorting through a handpiece.

While shown with six applicators 208a-208f in FIG. 2, the number of applicators used in the treatment system 200 may vary. For example, the system 200 may utilize one, two, three, four, five, or seven or more applicators. The number of applicator switches 206a-206f and return switches 210a-210a may also vary based on the number of applicators used in the treatment system 200.

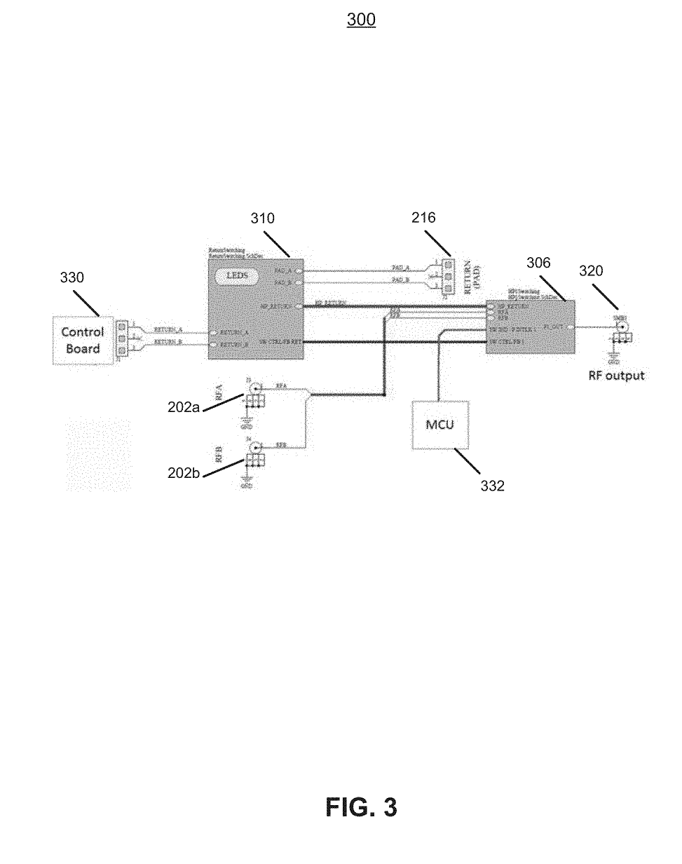

FIG. 3 illustrates an example circuit diagram 300 for use with an applicator, such as the applicators 208a-208f of FIG. 2. The circuit diagram 300 includes a handpiece switching circuit 306 and a return switching circuit 310. The handpiece switching circuit 306 is electrically connectable to the energy sources 202a and 202b and the return switching circuit 310. The handpiece switching circuit 306 can switch between the energy sources 202a-202b or the return switching circuit 310. A microcontroller unit (MCU) 332 may control the switching of the handpiece switching circuit 306. When the handpiece switching circuit 306 selects one of the energy sources 202a-202b, a corresponding applicator 208a-208f (as shown in FIG. 2) is electrically connected to the selected energy source 202a or 202b and receives energy from the selected energy source 202a or 202b. The applicator 208a-208f then emits energy from the RF output 320. When the handpiece switching circuit 306 selects the return switching circuit 310, the corresponding applicator 208a-208f is disconnected from both energy sources 202a and 202b and is instead electrically connected to the return switching circuit 310. Selecting the return switching circuit 310 allows an applicator 208a-208f to be electrically connected to ground. When an applicator 208a-208f is connected ground, the applicator 208a-208f can act as a return path for energy being applied to a patient.

The return switching circuit 310 is electrically connectable to ground (not shown). In some embodiments, the return switching circuit 310 is electrically connectable to a return pad, such as the return pad 216 of FIG. 2. A control board 330 may control the operation of the return switching circuit 330. The return switching circuit 310 may also receive control signals from the MCU 332 via the handpiece switching circuit 332. When the return switching circuit 310 is electrically connected to an applicator 208a-208f as described above (e.g., when the applicator acts as a return path for energy being applied to a patient), the return switching circuit 310 provides an electrical connection to ground for the applicator 208a-208f. When the return pad 216 acts as the return path for energy being applied to the patient, the return switching circuit 310 provides an electrical connection to ground for the return pad 216.

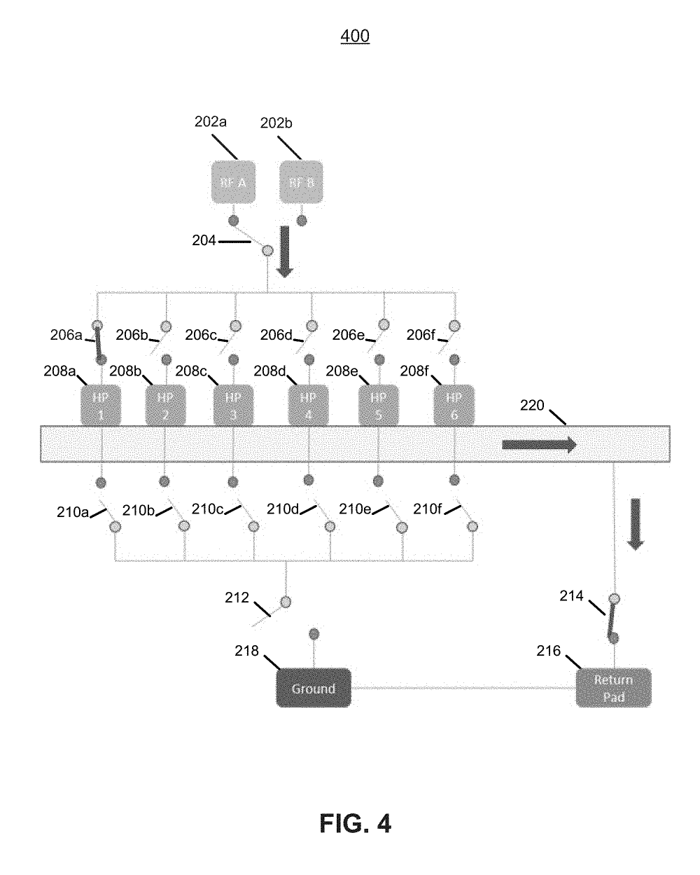

FIG. 4 illustrates an example switching diagram for the treatment system 400 when energy is flowing through the system 400. The switching diagram of FIG. 4 is the same as the switching diagram of FIG. 2, but source switch 204 is now selecting energy source 202a (“RF A”), and applicator switch 206a and return pad switch 214 are now closed. Thus, applicator 208a (“HP 1”) is electrically connected to energy source 202a. Energy flows from the energy source 202a to the applicator 208a and into a treatment area located under applicator 208a of the patient's body 220. A return pad 216 is attached to the patient's body 220 and allows the energy to flow from the treatment area of the patient's body 220 to ground 218 by closing the return pad switch 214. After energy is applied to the treatment area by applicator 208a, the area under applicator 208a is treated, and the applicator 208a may be disconnected from the energy source 202a by opening applicator switch 206a. Then, another applicator 208b-208f may be connected to the energy source 202a by closing its corresponding applicator switch 206b-206f. The next applicator 208b-208f then applies energy to a different treatment area of the patient's body 220. The system 200 sequentially provides energy to each applicator 208a-208f so that different treatment areas of the patient's body 220 are treated with energy at different times.

In some embodiments, each applicator 208a-208f receives energy from one energy source (e.g., energy source 202a). In other embodiments, different applicators 208a-208f receive energy from different energy sources 202a or 202b (e.g., applicator 208a receives energy from energy source 202a, applicator 208b receives energy from energy source 202b, applicator 208c receives energy from energy source 202a, and so on). In still other embodiments, each applicator 208a-208f alternately receives energy from both energy sources 202a and 202b (e.g., applicator 208a receives energy from energy source 202a for a first period of time, and then receives energy from energy source 202b for a second period of time, and likewise for each applicator 208a-208f).

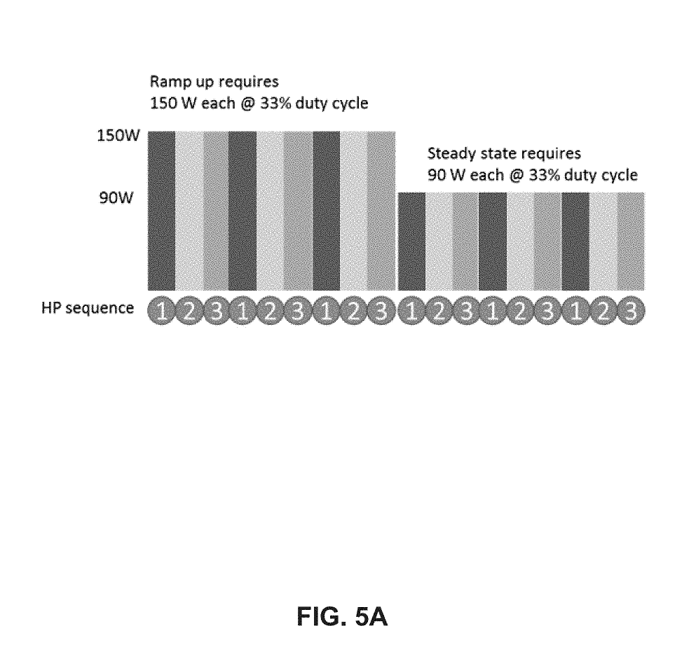

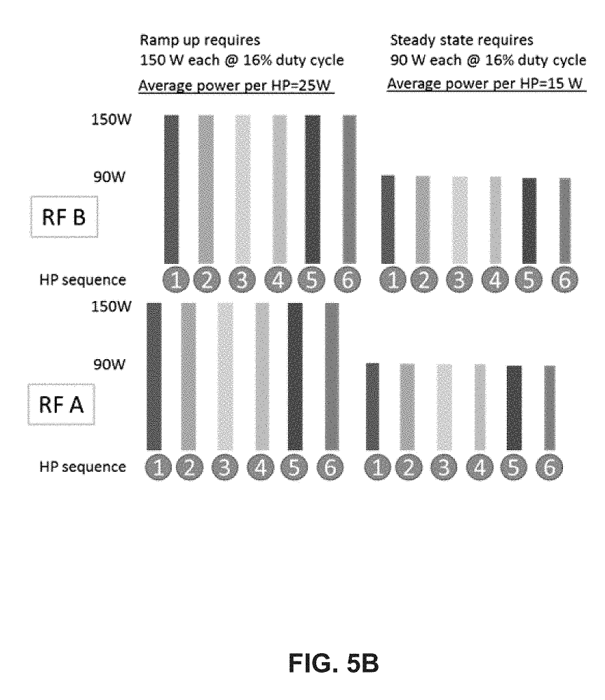

FIG. 5A illustrates an example energization pattern of how energy may be provided to three different applicators, such as applicators 208a-208c of FIG. 4. As shown in FIG. 5A, applicator “1” is initially provided with a warm up power level (e.g., 150 W) for a predetermined period of time. Then applicator “2” is provided with the warm up power level for the predetermined time, followed by applicator “3”. Each of the applicators “1”, “2”, and “3” may be provided with energy by either energy source 202a or 202b of FIG. 4. Sequentially providing applicators “1”, “2”, and “3” with the warm up power level for the predetermined period of time is repeated until the tissue being treated by each of the applicators reaches a target temperature (for example, as shown in FIG. 5A, each applicator receives 150 W of energy over three different predetermined periods of time). In some embodiments, the target skin temperature is 45° C. for which the fat temperature may be about 47° C., which is sufficient to damage fat cells by apoptosis. A nominal target range for the fat treatment temperature is 43° C. to 47° C., preferably 45° C. to 47° C. A study was conducted that determined at temperatures below 47° C., heated fat tissue cools at a rate of 1° C. to 3° C. per minute when the heat source is removed. Thus, the predetermined periods of time are set so that the temperature of any particular portion of the fat tissue does not drop by more than a threshold amount during the warm up period. In some embodiments, this threshold is 2° C. In some embodiments, this threshold is 1° C. In some embodiments, this threshold is 0.5° C. In some embodiments, the predetermined periods of time during the warm up period are less than 180 seconds. In some embodiments, the predetermined periods of time during the warm up period are less than 120 seconds. In some embodiments, the predetermined periods of time during the warm up period are less than 60 seconds.

After the tissue being treated reaches the target temperature, the power provided to each applicator is decreased to a nominal power level (e.g., 90 W) to maintain the tissue at the target temperature. The nominal power level is then sequentially provided to each applicator “1”, “2”, and “3” for predetermined periods of time until treatment with the applicators is complete. At temperatures above 47° C., heated fat tissue cools at a rate of 2° C. to 3° C. per minute when the heat source is removed. Thus, the predetermined periods of time are set so that the temperature of any particular portion of the fat tissue does not drop by more than a threshold amount during the “maintenance” (therapy) period. In some embodiments, this threshold is 2° C. In some embodiments, this threshold is 1° C. In some embodiments, this threshold is 0.5° C. In some embodiments, the predetermined periods of time during the maintenance period are less than 60 seconds. In some embodiments, the predetermined periods of time during the maintenance period are less than 45 seconds. In some embodiments, the predetermined periods of time during the maintenance period are less than 30 seconds.

In one example, the warm up, time-averaged power level provided to any particular applicator to ramp up the temperature of tissue being treated is 50 W. The time-averaged power level provided to the applicators to maintain the tissue at a target temperature is 30 W. When an interleaving energization pattern (such as shown in FIG. 5A) is applied to a group of applicators, each applicator will receive peak power of:

$$Peak Power = \frac{Required Power}{Duty Cycle} = Required Power \times Number of HPs \qquad ( Eqn . 1 )$$

The right-hand side expression is calculated based upon an equal duty cycle among the energized applicators.

By sequentially providing energy to each applicator as shown in FIG. 5A, the continuous energy at nominal average power is applied across a combined treatment area of the patient. In this programmed energization pattern, the target tissue of the patient receives longer treatment duration in average during a nominal treatment duration, compared with a treatment plan of energizing one single treatment area independently for the same treatment duration.

In some embodiments, the warm up process is a step function from zero power to a nominal warm up power level. In other embodiments, the warm up process is controlled by a feedback mechanism using the nominal warm up power level as a setpoint. In some embodiments, the feedback mechanism is a proportional-integral-derivative (PID) controller. In some embodiments, the feedback mechanism is a quasi-PID controller. In some embodiments, the coefficients of the PID or quasi-PID controller are determined from measurements of the treatment area of the patient's body. In some embodiments, one or more coefficients of the PID or quasi-PID controller are set to zero.

FIG. 5B illustrates an example energization pattern of how energy may be alternately provided to six different applicators, such as applicators 208a-208f of FIG. 4. As shown in FIG. 5B, applicator “1” is initially provided with a warm up power level (e.g., 150 W) from a first energy source “RF A” (e.g., energy source 202a of FIG. 4) for a first period of time. Then the energy source is switched to a second energy source “RF B” (e.g., energy source 202b of FIG. 4) and applicator “1” is provided with the warm up power level (e.g., 150 W) from the second energy source “RF B” for a second period of time. The same pattern of providing the warm up power level from the first energy source “RF A” followed by providing the warm up power level from the second energy source “RF B” is repeated for each applicator “1” through “6” until the tissue being treated by each of the applicators reaches a target temperature. After the tissue being treated reaches the target temperature, the energy provided to each applicator by each energy source is decreased to a nominal power level (e.g., 90 W) to maintain the tissue at the target temperature. The nominal power level is then alternately provided by each energy source “RF A” and “RF B” to each applicator “1” through “6” until treatment with the applicators is complete. For example, applicator “1” receives the nominal power level from energy source “RF A” for a first period of time. Then the energy source is switched to a second energy source “RF B” and applicator “1” is provided with the nominal power level from the second energy source “RF B” for a second period of time. The same pattern of providing the nominal power level from the first energy source “RF A” followed by providing the nominal power level from the second energy source “RF B” is repeated for each applicator “1” through “6” until treatment with the applicators is complete.

Compared to the energization pattern of FIG. 5A, the energization pattern of FIG. 5B energizes the same applicator using two duty cycles consecutively, one from energy source “RF A” (e.g., energy source 202a of FIG. 4) and the other from energy source “RF B” (e.g., energy source 202b of FIG. 4). In one example, energy source “RF A” is selected to provide the energy to an applicator at first with 50% of the energization. Then energy source “RF B” is selected to provide energy to the same applicator with another 50% of the energization. Thus, each energy source is required to supply only 50% of the nominal required average power for a given applicator. In this way, the energization pattern of FIG. 5B takes advantage of the high thermal constant of the tissue heating and optimizes the thermal management and power requirements of the applicators over multiple energy sources.

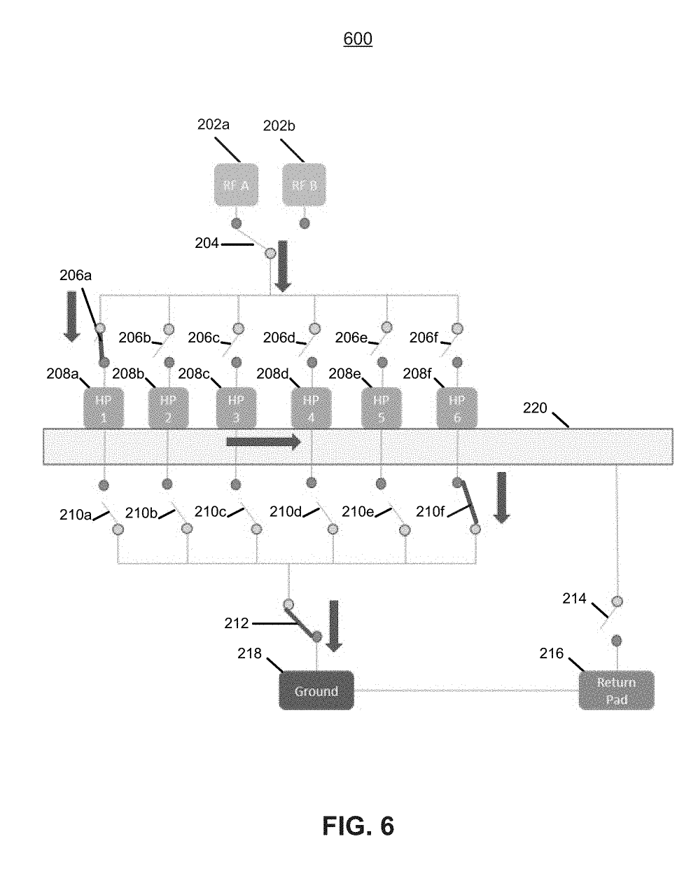

FIG. 6 illustrates another example switching diagram for the treatment system 600 when energy is flowing through the system 600. The switching diagram of FIG. 6 is the same as the switching diagram of FIG. 2, but source switch 204 is now selecting energy source 202a (“RF A”), and applicator switch 206a, return switch 210f, and ground switch 212 are now closed and switch 214 is open. Thus, applicator 208a (“HP 1”) is electrically connected to energy source 202a. Energy flows from the energy source 202a to the applicator 208a and into a treatment area of the patient's body 220. A second applicator 208f (“HP 6”) is electrically connected to ground 218 by the closing of return switch 210f and ground switch 212. Thus, energy flows from applicator 208a through the patient's body 220 to applicator 208f, and then to ground 218. In this way, the areas underneath two applicators (e.g. 208a and 208f) are treated simultaneously with the energy flow from a single handpiece. In some embodiments, the two paired applicators do not have side edges that are adjacent to each other. Otherwise, the electric current would travel from one applicator to the other through the skin without heating up the fat. At most, electrically paired applicators are placed diagonally to each other where exactly one corner of one applicator is near a corner of the other applicator of the pair.

The advantage of this approach is it reduces the required peak power by one half which reduces the power handling requirements of the energy sources and increases the system efficiency:

Peak Power=½×Required Power×Number of HPs (Eqn. 2)

This approach has the added advantage of eliminating the need for a return pad, which increases system complexity and may limit the maximum total treatment power (and therefore treatment area) for a single treatment.

After energy is applied to the patient by flowing energy through applicators 208a and 208f, the applicator 208a may be disconnected from the energy source 202a by opening applicator switch 206a, and the applicator 208f may be disconnected from ground 218 by opening return switch 210f. Then, another pair of applicators 208b-208f may be selected for applying energy to the patient. For example, applicator 208b (“HP 2”) may be electrically connected to the energy source 202a by closing its corresponding applicator switch 206b, and applicator 208e (“HP 5”) may be electrically connected to ground 218 by closing its corresponding return switch 210e. The applicator 208b then applies energy to a different treatment area of the patient's body 220 and the energy flows to ground through applicator 210e. The system 600 sequentially provides energy to different pairs of applicator 208a-208f so that different treatment areas of the patient's body 220 are treated with energy at different times.

In some embodiments, each pair of applicator 208a-208f receives energy from one energy source (e.g., energy source 202a). In other embodiments, different pairs of applicators 208a-208f receive energy from different energy sources 202a or 202b (e.g., applicator 208a receives energy from energy source 202a, applicator 208b receives energy from energy source 202b, applicator 208c receives energy from energy source 202a, and so on). In still other embodiments, each pair of applicator 208a-208f alternately receives energy from both energy sources 202a and 202b (e.g., applicator 208a receives energy from energy source 202a for a first period of time, and then receives energy from energy source 202b for a second period of time, and likewise for each pair of applicator 208a-208f).

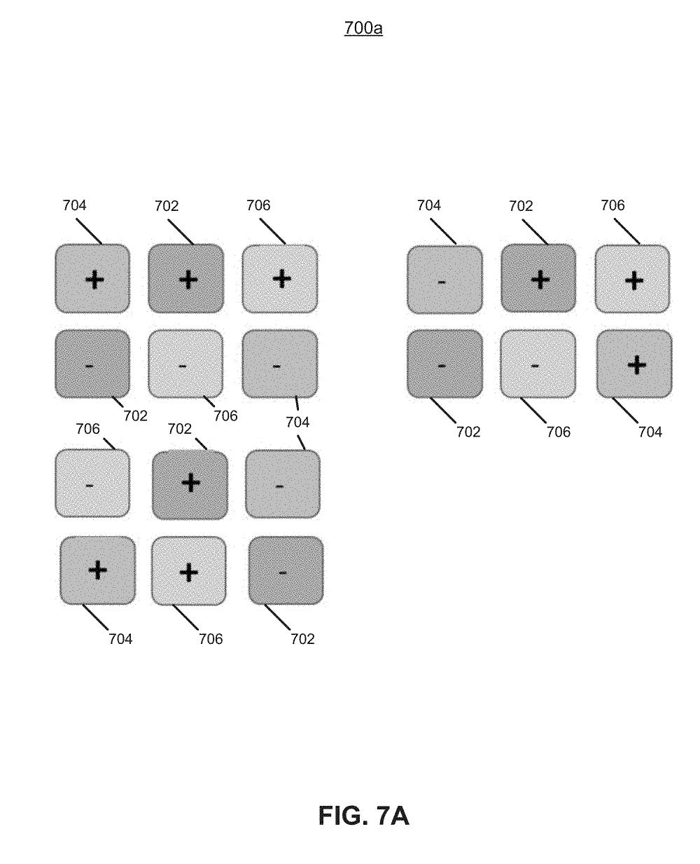

FIGS. 7A and 7B illustrate different arrangements of applicators that can be used when applying energy to treatment areas. In such arrangements, the applicators are grouped into pairs of applicators. As shown in FIG. 7A, there are three sets of six applicators, where the six applicators in each set are grouped into pair 702, pair 704, and pair 706. Energy is applied to pairs of applicators 702, 704, and 706, such as described in reference to FIG. 6, where the energy flows from one applicator, through the patient's body, and then exits through a second applicator. In other words, the applicators are paired to form a current loop. In the examples shown in FIGS. 7A and 7B, three pairs of applicators 702, 704, and 706 are used to treat a region of the patient's body. Energy flows from a “+” applicator in a pair of applicators to a “−” applicator in the pair of applicators. For example, the “+” applicator in applicator pair 702 may be connected to energy source 202a of FIG. 6 and the “−” applicator in applicator pair 702 may be connected to ground 218 of FIG. 6. The applicators in each pair of applicators 702, 704, and 706 are spaced at a distance from each other that allows for sufficient heat absorption depth into the patient's tissue. In this embodiment, no electrically paired applicators have side edges that are adjacent to each other. Otherwise, the electric current would travel from one applicator to the other through the skin without heating up the fat. At most, electrically paired applicators are placed diagonally to each other where exactly one corner of one applicator is near a corner of the other applicator of the pair, as demonstrated by pairs 702 and 706 in FIG. 7A.

The different arrangements of applicators shown in FIGS. 7A and 7B allow for regions of the patient's body of similar sizes and shapes to be treated. In particular, FIG. 7B illustrates arrangements of six applicators comprising one row of two applicators and another row of four applicators. This may be used, for example, in treating the abdomen of a patient, where the row of four applicators are applied across the lower stomach, where there is a larger area to treat, and the row of two applicators are applied higher on the abdomen, where there is comparatively less fat to treat.

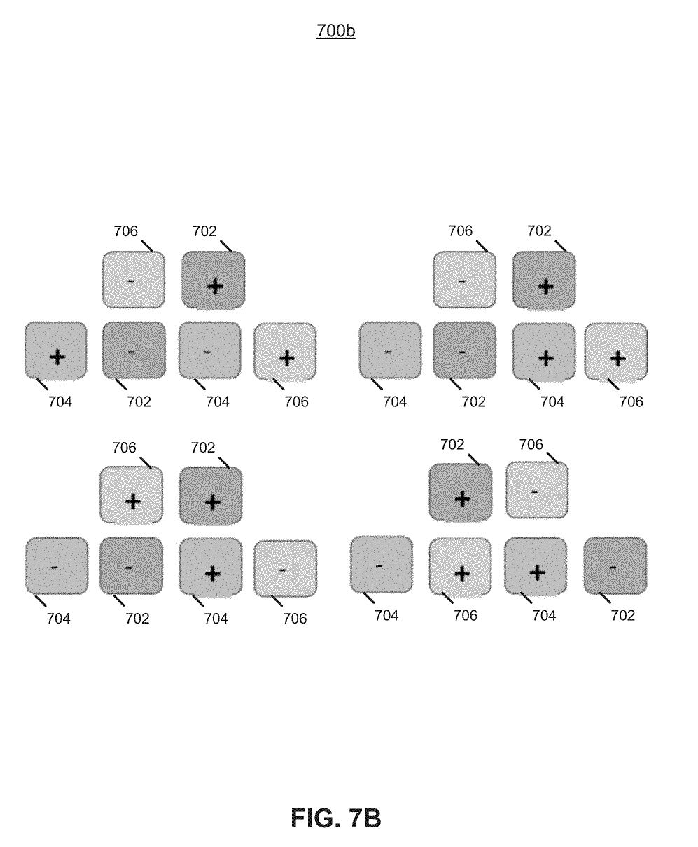

FIG. 8 illustrates an example energization pattern of how energy may be provided to three pairs of applicators, such as applicators 208a-208f of FIG. 6. As shown in FIG. 8, applicator “1” is initially provided with a warm up power level (e.g., 150 W) for a predetermined period of time. The warm up energy flows from applicator “1”, through the patient's body, and exits through applicator “4”. When the warm up energy exits through applicator “4”, applicator “4” effectively receives energy at approximately the same time as applicator “1”, as shown in FIG. 8. Thus, different pairs of applicators are effectively energized approximately simultaneously. After applicators “1” and “4” are energized with the warm up power level, applicators “2” and “5” are energized, followed by applicators “3” and 6”. Each of the applicators “1”, “2”, and “3” may be provided with energy by either energy source 202a or 202b of FIG. 6. Sequentially energizing pairs of applicators with the warm up power level for the predetermined period of time is repeated until the tissue being treated by each of the applicators reaches a target temperature (for example, as shown in FIG. 8, each applicators “1”, “2”, and “3” receive 150 W of energy over three different predetermined periods of time, which also energizes applicators “4”, “5”, and “6” during the three periods of time). After the tissue being treated reaches the target temperature, the energy provided to the applicators is decreased to a nominal power level (e.g., 90 W) to maintain the tissue at the target temperature. The nominal power level is then used to sequentially energize each pair of applicators until treatment with the applicators is complete.

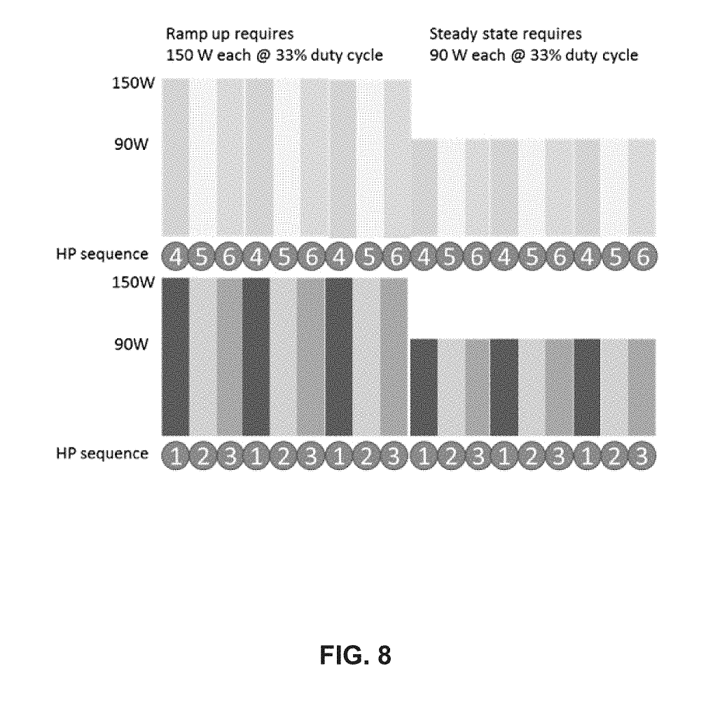

FIG. 9 illustrates another example switching diagram for the treatment system 900 when energy is flowing through the system 900. Instead of including a source switch as shown in FIG. 2, each of the applicator switches 206a-206b of FIG. 9 are now able to select between energy source 202a (“RF A”), energy source 202b (“RF B”), or no connection. As shown in FIG. 9, applicator switch 206a is selecting energy source 202a and applicator switch 206d is selecting energy source 202a. Thus, applicator 208a (“HP 1”) is electrically connected to energy source 202a and applicator 208d (“HP 4”) is electrically connected to energy source 202b. Energy flows from the energy source 202a to the applicator 208a and into a treatment area of the patient's body 220. At the same time, energy flows from the energy source 202b to the applicator 208d and into a different treatment area of the patient's body 220. A return pad 216 is attached to the patient's body 220 and allows the energy to flow from the two treatment areas of the patient's body 220 to ground 218 by closing the return pad switch 214. After energy is applied to the two treatment areas by applicators 208a 208d, the applicators 208a and 208d may be disconnected from the energy sources 202a and 202b by opening applicator switch 206a and 206d. Then, another pair of applicators may be connected to the energy sources 202a and 202b by selecting the energy sources 202a-202b with the applicator switches 206a-206f. The next pair of applicators then apply energy to two more treatment areas of the patient's body 220. The system 200 sequentially provides energy to different pairs of applicator 208a-208f so that different treatment areas of the patient's body 220 are treated with energy at different times.

In some embodiments, the energy sources 202a and 202b produce energy with different phase angles. In some embodiments, the energy sources 202a and 202b are about 180 degrees out of phase with each other. In this regard, about 180 degrees would encompass a range of 170 degrees to 190 degrees out of phase. In these embodiments, when two different applicators are electrically connected to each energy source 202a and 202b as described in reference to FIG. 9, electrical current can flow from energy source 202a, through an applicator, and then return through another applicator connected to the other energy source 202b. The direction of the current flow may alternate between the two connected applicators when the energy sources 202a and 202b output sinusoidal energy waves with opposite or approximately opposite phases. The closer the phase difference is to 180 degrees, the smaller the residual current. Any residual current will be passed to ground 218 through the return pad 216. In some embodiments, if all current is expected to pass from one connected applicator to the connected applicator, a system without a return pad is possible. This would require each applicator to pass the same current, however, while the presence of an external return pad allows for independent current control of each applicator. In one example, the current flowing through the return pad 216 may be half the amount as compared to the current that would flow through the return pad 216 when both energy sources 202a and 202b are in phase (such as in FIG. 9). This may prevent the return pad 216 from being overloaded with too much current and thus overheating, causing discomfort for the patient. A similar principle may apply to the embodiment shown in FIG. 6.

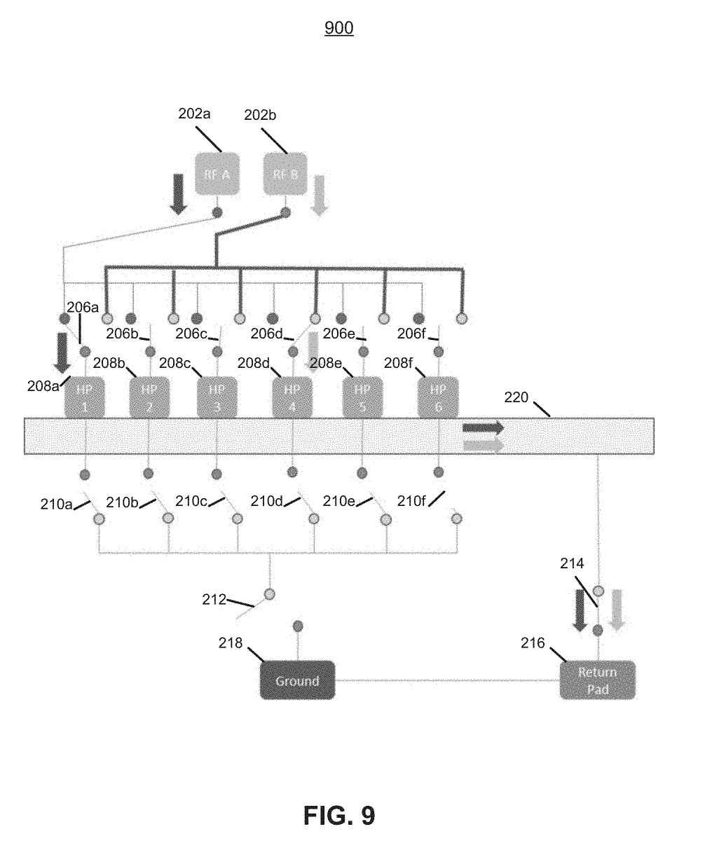

FIG. 10 illustrates an example energization pattern of how energy may be provided simultaneously to different pairs of applicators, such as applicators 208a-208f of FIG. 9. As shown in FIG. 10, applicator “1” is initially provided with a warm up power level (e.g., 150 W) from a first energy source “RF A” (e.g., energy source 202a of FIG. 9) for a predetermined period of time. At approximately the same time, applicator “4” is also provided with a warm up power level (e.g., 150 W) from a second energy source “RF B” (e.g., energy source 202b of FIG. 9) for the predetermined period of time. Then applicators “2” and “5” are provided with the warm up power level from the two energy sources for the predetermined time, followed by applicators “3” and “6”. Sequentially providing each pair of applicators “1” and “4”, “2” and “5”, and “3” and “6” with the warm up power level for the predetermined period of time is repeated until the tissue being treated by each of the applicators reaches a target temperature (for example, as shown in FIG. 10, each applicator receives 150 W of power over three different predetermined periods of time). After the tissue being treated reaches the target temperature, the energy provided to each applicator is decreased to a nominal power level (e.g., 90 W) to maintain the tissue at the target temperature. The nominal power level is then sequentially provided to each pair of applicator “1” and “4”, “2” and “5”, and “3” and “6” by the two energy sources until treatment with the applicators is complete.

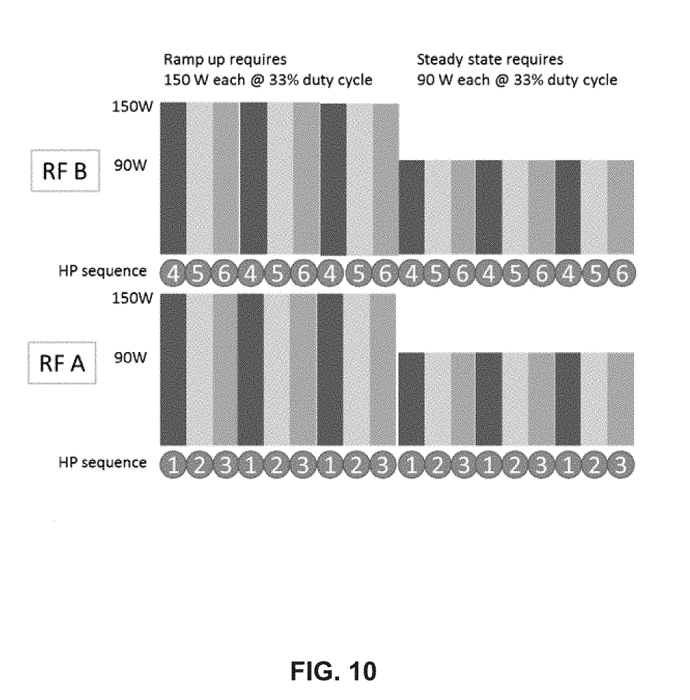

FIG. 11 illustrates an exemplary interleaving energization pattern (or timing sequence) with a constant duty cycle and variable individual application time. The first energy source (“RF card #1”) sequentially applies energy to handpieces 1, 2, and 3 at the same time that the second energy source (“RF card #2”) sequentially applies energy to handpieces 4, 5, and 6. In the initial time period T₁, energy is applied sequentially to each handpiece for 65 seconds for 1 cycle for a total period of 195 seconds. In the next time period T₂, energy is applied sequentially to each handpiece for 30 seconds for 1 cycle for a total period of 90 seconds. In the third time period T₃, energy is applied sequentially to each handpiece for 15 seconds for 1 cycle for a total period of 45 seconds. Finally, In the last time period T₄, energy is applied sequentially to each handpiece for 3 seconds for 64 cycles for a total period of 576 seconds, or 9 minutes and 36 seconds. T₁ and part of T₂ constitute the ramp up period, and the rest of T₂, T₃, and T₄, constitute the maintenance or treatment period. Thus, the ramp time is approximately 4 minutes long, followed by therapeutic period of approximately 12 minutes long.

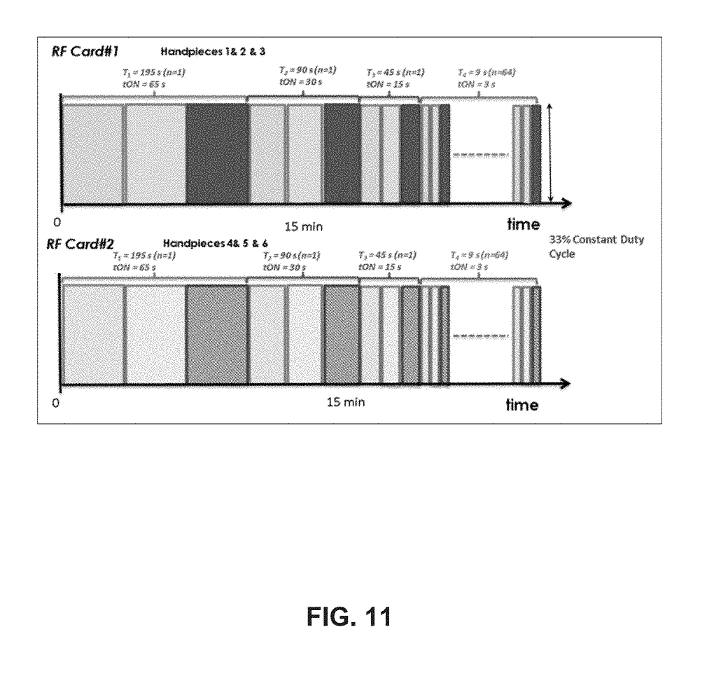

FIG. 12 illustrates the fat temperature progression 1200 during treatment using the interleaving energization pattern with a constant duty cycle and variable individual application time, as shown in FIG. 11, when performed in an in vivo experiment. The temperature of the fat tissue corresponding to handpiece 1 is monitored as the interleaving energization pattern for six handpieces shown in FIG. 11 is applied. Each handpiece has an application surface area of 40 cm². At the start 1211 of the ramp up phase, the fat tissue is at 37° C., human body temperature. When handpiece 1 is energized during time period T₁, the fat tissue reaches a temperature of nearly 45° C. by the time power is no longer applied to handpiece 1 at point 1213. The greatest temperature drop in time period T₁ is approximately 2° C. (from 45° C. to 43° C.) from point 1213 to point 1215, which corresponds to the time when power was not being applied to handpiece 1 and power was being applied to handpiece 2 and 3 (130 seconds).

After the second sequence begins at point 1215, the temperature of the fat surpasses the target fat treatment temperature of 45° C. at point 1217, at which point the process enters the treatment, or therapeutic, phase. During the time that power was not being applied to handpiece 1 and power was being applied to handpieces 2 and 3, the fat tissue experiences a 0.6° C. drop (in 60 seconds) in the second period, ending at point 1219. The fat tissue experiences a modest 0.3° C. drop (in 30 seconds) in the third period. During the therapy period period, the temperature of the fat is maintained about 45 degrees. At the end 1223 of therapy period, the temperature falls below the fat treatment temperature. When power is no longer being applied to any handpiece, the fat temperature 1225 falls towards human body temperature.

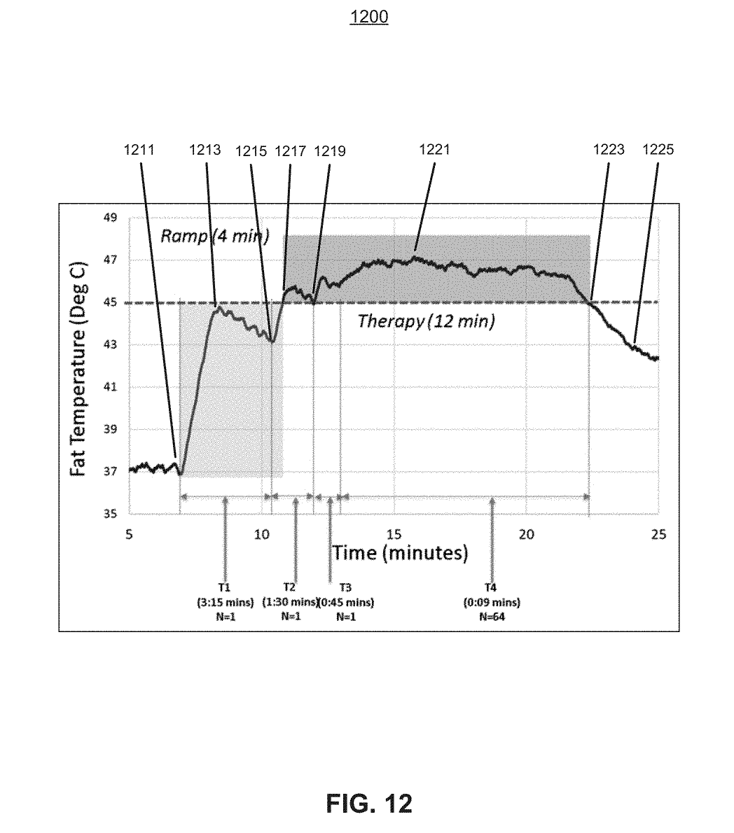

FIG. 13 illustrates an exemplary interleaving energization pattern (or timing sequence) with a variable duty cycle and variable individual application time. The first energy source (“RF card #1”) sequentially applies energy to handpieces 1, 2, 3, and 4. In the initial time period T₁, energy is applied to handpieces 1 and 2 (50% duty cycle) for 90 seconds each for 1 cycle for a total period of 180 seconds. In the second time period T₂, energy is applied to handpieces 1 and 2 (50% duty cycle) for 30 seconds each for 1 cycle for a total period of 60 seconds. In the third time period T₃, energy is applied to handpieces 1, 2, 3, and 4 (25% duty cycle) for 2 seconds each for 30 cycles for a total period of 240 seconds (4 minutes). In the fourth and final time period T₄, energy is applied to handpieces 1, 2, and 3 (33% duty cycle) for 3 seconds each for 64 cycles for a total period of 576 seconds, or 9 minutes and 36 seconds.

At the same time, the second energy source (“RF card #2”) sequentially applies energy to handpieces 3, 4, 5, and 6. In this embodiment, the pattern for the second energy source follows a separate set of time periods compared to the pattern for the first energy source. In the initial time period T₁, energy is applied to handpieces 3 and 4 (50% duty cycle) for 90 seconds each for 1 cycle for a total period of 180 seconds. In the second time period T₂, energy is applied to handpieces 3 and 4 (50% duty cycle) for 30 seconds each for 1 cycle for a total period of 60 seconds. In first half T_{3,A} of the third time period T₃, energy is applied to handpieces 5 and 6 (50% duty cycle) for 90 seconds each for 1 cycle for a total period of 180 seconds (3 minutes). Note that during this time period, the first energy source is providing energy to handpieces 1, 2, 3 and 4. In the second half T_{3,B} of the third time period T₃, energy is applied to handpieces 5 and 6 (50% duty cycle) for 30 seconds each for 1 cycle for a total period of 60 seconds. In the fourth and final time period T₄, energy is applied to handpieces 4, 5, and 6 (33% duty cycle) for 3 seconds each for 64 cycles for a total period of 576 seconds, or 9 minutes and 36 seconds. During this last time period, the first energy source applied energy to handpieces 1, 2 and 3. As can be seen from this embodiment, the particular energy source used to energize a handpiece can vary during the treatment.

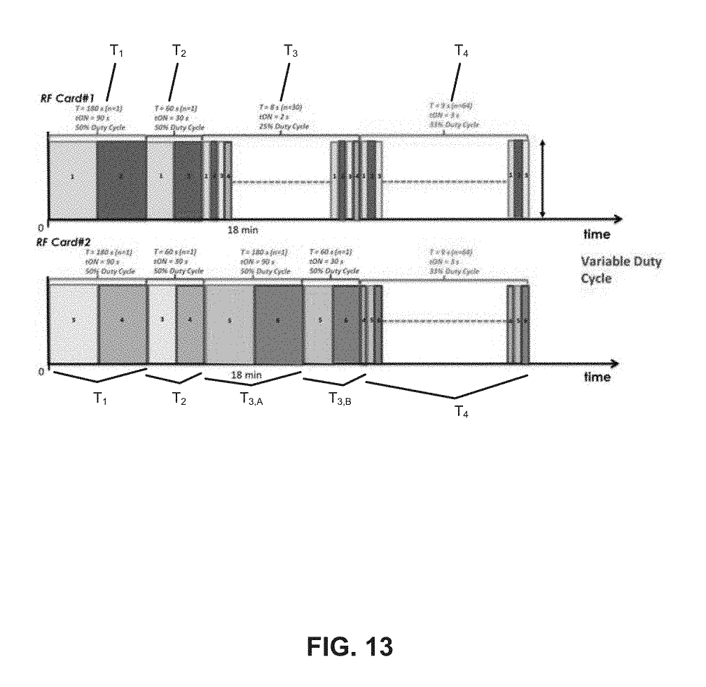

FIG. 14 illustrates an exemplary interleaving energization pattern (or timing sequence) with a constant duty cycle and constant individual application time. The first energy source (“RF card #1”) sequentially applies energy to handpieces 1, 2, and 3 at the same time that the second energy source (“RF card #2”) sequentially applies energy to handpieces 4, 5, and 6, where each energy is applied to each handpiece for the same amount of time throughout the ramp up and treatment. In some embodiments, that individual application time ranges from 2 to 8 seconds. The cycle is repeated until the total ramp up and treatment time is 12 to 15 minutes.

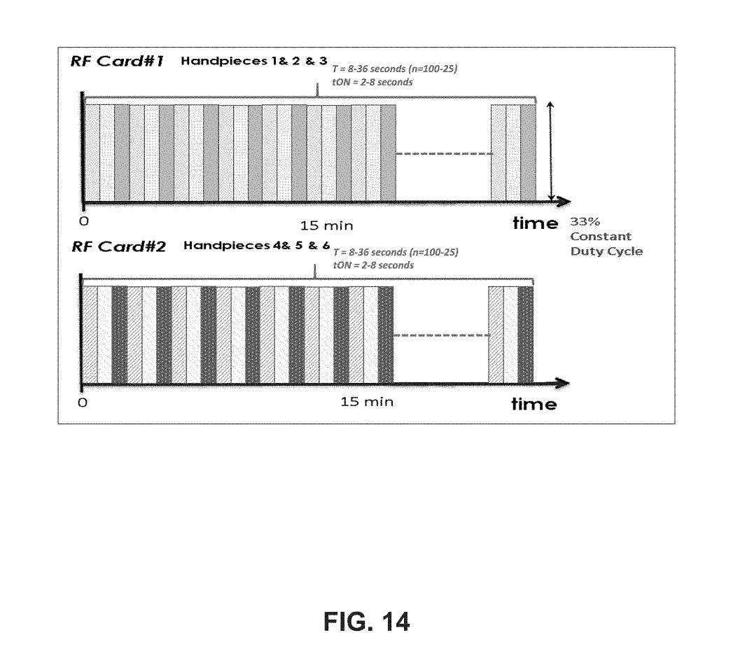

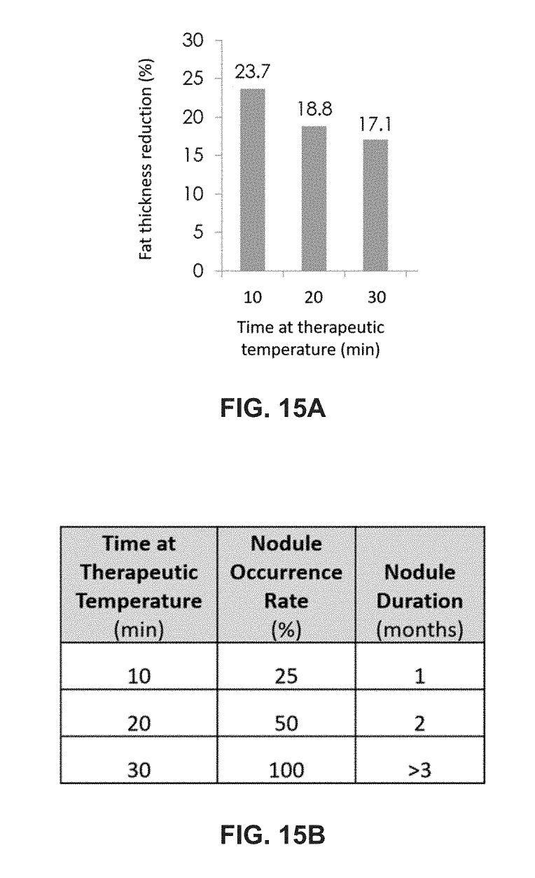

FIG. 15A illustrates the reduction in the fat layer thickness for various therapeutic exposure times as measured in a clinical study, and FIG. 15B illustrates the occurrence rate and duration of nodule formation in the fat layer for various therapeutic exposure times as measured in the same clinical study. The clinical study implemented embodiments where the skin temperature was maintained at a lower temperature than the fat without actively cooling the skin surface. In such embodiments, perfusion in the skin tissue and the thermal mass of the handpiece serve to cool the skin during treatment and maintain the skin's temperature at a sub-therapeutic level that is below the fat temperature. This “temperature inversion” allows for selective damage to the fat layer. As stated above, one goal of the present disclosure is to minimize treatment time without reducing efficacy or increasing patient discomfort. Therefore, it is also important to determine the optimum duration for which the fat tissue should be held within the therapeutic temperature range. A period that is too short will result in under treatment and lower efficacy. A period that is too long will result in over treatment that may trigger tissue inflammatory responses that reduce or limit efficacy while increasing discomfort and treatment time. In general, the temporal and three dimensional spatial temperature distribution in the fat and surrounding skin and muscle tissues determines the efficacy, selectivity, and discomfort of the treatment. Since this distribution is unique to the frequency, power, and exposure area of the energy source used and the cooling modality (active or passive) and rate provided by the applicator, a need exists to establish the optimum time at therapeutic temperature for the present invention that maximizes efficacy while minimizing treatment time and discomfort. Active cooling may be achieved using a water-cooled heat exchanger or thermoelectric cooler to extract heat from the skin surface through the applicator contact surface. Passive cooling relies on natural conduction and convection to extract heat from the skin through handpiece contact surface.

The clinical study evaluated the efficacy as a function of time at the therapeutic temperature for the preferred embodiment that uses an applicator with passive cooling. In the case of fat reduction, the goal is to achieve a reduction in the fat layer thickness of at least 15% and preferably greater than 20% for a single treatment as measured at about 3 months after treatment. FIG. 15A shows the reduction in the fat layer thickness as measured using ultrasound images for therapeutic exposure times of 10 minutes, 20 minutes, and 30 minutes using a 40 cm² applicator and an energy source generating energy at 2 MHz. In this study the flanks and abdomens of 30 patients were treated and total body weight was maintained within 4 lbs. The reduction in the fat layer thickness as shown in FIG. 15A is the average reduction in the fat layer thickness across the 30 patients.

FIG. 15B illustrates the occurrence rate and duration of nodule formation in the fat layer for various therapeutic exposure times as measured in the same clinical study. Nodules are inflamed fibrous tissue that result from extended hyperthermia. A high occurrence rate (>50%) and a high duration (>3 months) are signs of over-treatment, which may limit reduction in the treated fat layer thickness. The data clearly indicates that increasing the time at therapeutic temperature beyond 20 minutes begins to reduce the efficacy. For a 30-minute therapy time, nodule formation occurred in 100% of patients and the nodules had not resolved even after 3 months following treatment. It is also known in the prior art that the fat thickness reduction for a therapeutic time of 3 minutes to 4 minutes is approximately 11%. Therefore, to achieve maximum efficacy and minimize the treatment time, the therapeutic time should be maintained between 6 minutes and 25 minutes, or more preferably between 8 minutes to 20 minutes.

It is understood that the specific order or hierarchy of blocks in the processes/flowcharts disclosed is an illustration of exemplary approaches. Based upon design preferences, it is understood that the specific order or hierarchy of blocks in the processes/flowcharts can be rearranged. Further, some blocks can be combined or omitted. The accompanying method claims present elements of the various blocks in a sample order, and are not meant to be limited to the specific order or hierarchy presented.

The previous description is provided to enable any person skilled in the art to practice the various examples described herein. Various modifications to these examples will be readily apparent to those skilled in the art, and the generic principles defined herein can be applied to other examples. Thus, the claims are not intended to be limited to the examples shown herein, but are to be accorded the full scope consistent with the language of the claims, wherein reference to an element in the singular is not intended to mean “one and only one” unless specifically so stated, but rather “one or more.” The word “exemplary” is used herein to mean “serving as an example, instance, or illustration.” Any example described herein as “exemplary” is not necessarily to be construed as preferred or advantageous over other examples. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and can include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” can be A only, B only, C only, A and B, A and C, B and C, or A and B and C, where any such combinations can contain one or more member or members of A, B, or C. All structural and functional equivalents to the elements of the various examples described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like cannot be a substitute for the word “means.” As such, no claim element is to be construed under 35 U.S.C § 112(f) unless the element is expressly recited using the phrase “means for.”

What is claimed is:

1. A system for treating an area of a patient comprising a plurality of subareas with energy, the system comprising:
one or more energy sources, wherein each energy source is configured to independently provide radiofrequency energy;
a plurality of energy applicators, numbering more than the number of energy sources, wherein each energy applicator is aligned with a different subarea and is configured to apply energy to the subarea when provided with energy from the one or more energy sources; and
a switching circuit configured to energize each energy applicator in the plurality of energy applicators with energy provided from the one or more energy sources using a predetermined pattern of energization, wherein the predetermined pattern of energization comprises:
a first phase lasting a first time period, wherein the energy sources sequentially provide energy to multiple applicators one or more times at a frequency and a first range of power levels to elevate temperatures of fat tissue in each subarea to a fat treatment temperature, wherein the temperature of fat tissue in a subarea does not fall more than 2 degrees Celsius during any time in the first time period when energy is not being applied to the subarea, and
a second phase lasting a second time period, wherein the energy sources sequentially and repeatedly provide energy to multiple applicators at a frequency and at a second range of power levels to maintain temperatures of fat tissue in each subarea at or above the fat treatment temperature, wherein the temperature of fat tissue in a subarea does not fall more than 2 degrees Celsius during any time in the second time period when energy is not being applied to the subarea.

2. The system of claim 1, wherein the temperature of fat tissue in a subarea does not fall more than 1 degree Celsius during any time in the first time period when energy is not being applied to the subarea.

3. The system of claim 1, wherein the temperature of fat tissue in a subarea does not fall more than 0.5 degree Celsius during any time in the second time period when energy is not being applied to the subarea.

4. The system of claim 1, wherein during the first time period, the time between consecutive applications of energy to each energy applicator is less than 180 seconds.

5. The system of claim 4, wherein during the first time period, the time between consecutive applications of energy to each energy applicator is less than 120 seconds.

6. The system of claim 5, wherein during the first time period, the time between consecutive applications of energy to each energy applicator is less than 60 seconds.

7. The system of claim 1, wherein during the second time period, the time between consecutive applications of energy to each energy applicator is less than 60 seconds.

8. The system of claim 7, wherein during the second time period, the time between consecutive applications of each energy applicator is less than 45 seconds.

9. The system of claim 8, wherein during the second time period, the time between consecutive applications of each energy applicator is less than 30 seconds.

10. The system of claim 1, wherein:
the plurality of applicators are grouped into 3 pairs of applicators,
the treatment area of the patient comprises 6 subareas,
each of 6 energy applicators is applied to each of the 6 subareas,
the first phase comprises repeatedly and sequentially applying energy to each pair of applicators, and
the second phase comprises repeatedly and sequentially applying energy to each pair of applicators.

11. The system of claim 10, wherein:
a first energy source is applied to the first of each pair of applicators;
a second energy source is applied to the second of each pair of applicators; and
the first energy source is between 170 degrees and 190 degrees out of phase with the second energy source.

12. The system of claim 11, wherein the first energy source is 180 degrees out of phase with the second energy source.

13. The system of claim 10, wherein one energy applicator of the pair of energy applicators is electrically connected as the current return path of the other energy applicator of the pair of energy applicators.

14. The system of claim 10, wherein the energy applicators in each pair of energy applicators are not adjacent to each other.

15. The system of claim 1, wherein the first time period is between 20 and 225 seconds.

16. The system of claim 1, wherein the second time period is between 9 minutes and 15 minutes.

17. The system of claim 1, wherein the frequency of the energy sources is between 200 kHz and 10 MHz.

18. The system of claim 17, wherein the frequency of the energy sources is between 1 MHz and 6.5 MHz.

19. The system of claim 18, wherein the frequency of the energy sources is between 1 MHz and 3 MHz.

20. The system of claim 19, wherein the frequency of the energy sources is about 2 MHz.

21. The system of claim 1, wherein the fat treatment temperature is between 43 degrees Celsius and 47 degrees Celsius.

22. The system of claim 21, wherein the second time period is between 6 minutes and 25 minutes.

23. The system of claim 22, wherein the second time period is between 8 minutes and 20 minutes.

24. The system of claim 1, wherein each subarea has a surface area between 20 square cm and 80 square cm.

25. A system for treating a treatment area of a patient comprising a plurality of subareas with energy, the system comprising:
an energy source configured to provide radiofrequency energy;
a plurality of energy applicators, wherein:
the plurality of energy applicators are arranged in a grid-like array;
each energy applicator is aligned with a different subarea and is configured to apply energy to the subarea when provided with energy from the energy source;
each energy applicator is paired with another energy applicator in the plurality of energy applicators wherein no pair of energy applicators comprises energy applicators that are aligned with subareas whose side edges are adjacent to each other; and
a switching circuit configured to energize each energy applicator in the plurality of energy applicators with energy from the energy source using a predetermined pattern of energization, wherein the predetermined pattern of energization comprises:
sequentially providing energy to two or more successive pairs of the energy applicators one at a time, wherein when an energy applicator of a pair of energy applicators is provided with energy, the other energy applicator of the pair of energy applicators is acting as a current return.

26. A method for treating an area of a patient comprising a plurality of subareas with energy, the method comprising:
energizing each energy applicator in a plurality of energy applicators with energy provided from one or more energy sources, wherein:
the plurality of energy applicators numbers more than the number of energy sources;
each energy applicator is aligned with a different subarea and is configured to apply energy to the subarea when provided with energy from the one or more energy sources; and
each energy source is configured to independently provide radiofrequency energy, using a predetermined pattern of energization, wherein the predetermined pattern of energization comprises:
a first phase lasting a first time period, wherein the energy sources sequentially provide energy to more than multiple applicators one or more times at a frequency and a first range of power levels to elevate temperatures of fat tissue in each subarea to a fat treatment temperature, wherein the temperature of fat tissue in a subarea does not fall more than 2 degrees Celsius during any time in the first time period when energy is not being applied to the subarea, and
a second phase lasting a second time period, wherein the energy sources sequentially and repeatedly provide energy to multiple applicators at a frequency and at a second range of power levels to maintain temperatures of fat tissue in each subarea at or above the fat treatment temperature, wherein the temperature of fat tissue in a subarea does not fall more than 2 degrees Celsius during any time in the second time period when energy is not being applied to the subarea.

27. The method of claim 26, wherein:
the plurality of applicators are grouped into 3 pairs of applicators,
the treatment area of the patient comprises 6 subareas,
each of 6 energy applicators is applied to each of the 6 subareas,
the first phase comprises repeatedly and sequentially applying energy to each pair of applicators, and
the second phase comprises repeatedly and sequentially applying energy to each pair of applicators.

28. The method of claim 27, wherein:
a first energy source is applied to the first of each pair of applicators;
a second energy source is applied to the second of each pair of applicators; and
the first energy source is between 170 degrees and 190 degrees out of phase with the second energy source.

29. The method of claim 26, wherein the frequency of the energy sources is between 1 MHz and 3 MHz.

30. The method of claim 26, wherein the fat treatment temperature is between 43 degrees Celsius and 47 degrees Celsius.
